(19) United States
(12) Patent Application Publication
(10) Pub. No.: US 2007/0072246 A1
(43) Pub. Date: Mar. 29, 2007

(54) Cell-based assays for determining drug action

(76) Inventors: Ellen L. Berg, Palo Alto, CA (US); Jennifer Melrose, La Honda, CA (US); Eric J. Kunkel, San Mateo, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/570,081

(22) PCT Filed: Sep. 2, 2004
(86) PCT No.: PCT/US04/28970

§ 371 Date: Dec. 18, 2006

Related U.S. Application Data
(60) Provisional application No. 60/500,180, filed on Sep. 3, 2003.

(51) Int. Cl. G01N 33/567; G06F 19/00; C12N 5/08

(52) U.S. Cl. 435/7.2; 702/19; 435/366

(57) ABSTRACT

Compositions and methods are provided for the classification of biologically active agents according to their effect on human biology, through the use of complex, primary human cell-based disease models in scalable assay formats. The systems of the invention utilize the simultaneous activation of multiple signaling pathways to generate and identify patterns of expression of physiologically important cell surface and secreted molecules. Combinations of multiple cell types may be utilized. Systems encompassing multiple cell types not only respond to perturbations of each cell type's intracellular signaling networks, but also to inhibition of pathways of communication between cells. Readout information may be combined in multi-system analysis, where the profiles obtained from multiple systems are combined in order to provide enhanced resolution for agent classification.

### CROSS-REFERENCE TO RELATED APPLICATION

This application is related to copending U.S. patent application Ser. Nos. 10/236,558 and 10/220,999, both filed 5 Sep. 2002, and Ser. No. 09/800,605, filed 6 Mar. 2001, each of which is incorporated herein by reference.

### FIELD OF THE INVENTION

The present invention relates generally to the analysis of gene and drug function, the identification of biological pathways, and more particularly to methods for identifying and characterizing drugs by mechanism of action, defining signal transduction pathway architecture, finding relationships between signaling components, and identifying drug targets and drugs that affect those targets. The invention therefore relates to the fields of biology, molecular biology, chemistry, medicinal chemistry, pharmacology, and medicine.

### BACKGROUND OF THE INVENTION

Completion of the human genome sequence, in combination with modem approaches to chemical diversity, has opened up unparalleled opportunities for development of new medicines. Realization of this potential requires rapid and practical approaches to understanding the functions of molecules in the context of human biology. The discipline of systems biology has the potential to provide insights into human biology by modeling complex system responses based on the analysis of large scale measurements of molecular components. While some progress has been reported in model systems using yeast, the application of these large scale measurement approaches to human disease biology is less certain, given the greater complexity of cell and organ-level responses in human biology and disease.

Useful assays for measuring biological activities give robust and reproducible changes in a measurable parameter in the presence of a test article. In drug discovery, such assays are often either biochemical assays, such as kinase or enzyme assays, or cell-based assays designed to measure the activity of a specific target or pathway. Examples of cell-based assays include gene reporter assays, NFκB translocation assays, and the like. Features that distinguish typical cell based assays in drug discovery include an assay design that emphasizes the activity of a single specific target or pathway; use of a single cell type, typically a cell line; and the measurement of a single robust readout, e.g. a calcium signal, or the like.

However, while such assays can be highly sensitive and reproducible, they have several limitations, including the need to develop individual assays for each new target or pathway; specificity information of the test agent for the assay target is not provided; and information as to the mechanism of action of active agents in the assay is not provided. An improved assay format would remain robust and reproducible but also provide information on multiple targets and pathways in each assay, including information on the mechanism and specificity of a test agent for a target or pathway.

Such improved methodology has been described in PCT patent publication No. WO/167103, published 13 Sep. 2001, and U.S. patent application Ser. Nos. 10/236,558 and 10/220,999, both filed 5 Sep. 2002. These methods employ pattern recognition and mathematical modeling algorithms that enable the reconstruction of signal transduction networks from various types of gene expression and/or protein expression array data. While these methods enable the identification of drug targets and the mechanisms of drug action, there remains a need for improved methods that better reflect the complex biological environment of the human body as well as assays to determine whether a compound has the desired biological effects without undesired side effects, particularly with respect to drugs that have an anti-inflammatory effect. The present invention meets these and other needs.

### SUMMARY OF THE INVENTION

Compositions and methods are provided for the classification of biologically active agents according to their effect on human biology, through the use of complex, primary human cell-based disease models in scalable assay formats. The systems of the invention utilize the simultaneous activation of multiple signaling pathways to generate and identify patterns of expression (or “profiles”) of physiologically important cell surface and secreted molecules on primary cells in one or more complex environments, including environments simulating inflammation. The assay system is robust, reproducible, responsive to and discriminatory of the activities of a large number of agents, including biological factors, compounds, and genes.

In one embodiment of the invention, it is shown that biologically active agents induce characteristic response profiles in systems comprising primary human cells in complex, biologically-relevant environments, which profiles can be captured by measuring a relatively small number of physiologically significant protein readouts. Such readout parameters are selected for their information content and relevance to the physiological process of interest.

In another embodiment of the invention, combinations of multiple cell types are utilized, e.g. combinations of different primary cell types, primary cell types in combination with cell lines, etc. Systems encompassing multiple cell types not only respond to perturbations of each cell type's intracellular signaling networks, but also to inhibition of pathways of communication between cells. Thus, systems comprising multiple cell types can provide additional coverage of biological function space.

In another embodiment of the invention, readout information is combined in multi-system analysis. The profiles obtained from multiple systems are combined in order to provide enhanced resolution for agent classification.

### BRIEF DESCRIPTION OF THE FIGURES

FIG. 1. A system encompassing endothelial cells in a complex pro-inflammatory environment responds reproducibly to inhibitors of multiple signaling pathways. Endothelial cells were cultured with IL-1β, TNF-α and IFN-γ for 24 hours in the presence of anti-TNF-α (4 μg/ml), apigenin (6 μM), PD098059 (10 μM) or PD169316 (10 μM). Readout parameters were measured by ELISA as described in the Materials and Methods. Data are presented as log expression ratios (log₁₀[parameter value with drug/parameter value of control]) relative to solvent or media controls. The mean (black dash) and individual data points (red dots) are shown for each parameter (n=10-12 independent experiments). Individual data points represent assays performed on different days and/or with different endothelial cell donors.

FIG. 2. Drugs targeting common pathways and mechanisms induce similar system responses: drug comparison by “homology of function”. Endothelial cells were cultured with IL-1β, TNF-α and IFN-γ for 24 hours in the presence of the indicated drugs. (a) Heat map of log parameter expression ratios from individual experiments, showing the increase (green), decrease (red) or lack of change (black) of individual parameter levels. Color saturation reflects the magnitude of the drug effect (see scale, bottom). (b) Pearson correlations for pairwise comparisons of individual experimental profiles: positive correlations are in blue (most intense for r>0.9); black is no correlation (r˜0); and yellow indicates a negative correlation. A dendrogram showing the results of non-supervised hierarchical clustering of the log expression ratio profiles for each compound and experiment, using the Pearson correlation coefficient as the clustering metric, is presented at the right. The clustering results were used in a non-biased fashion to determine the order of presentation of compounds (a) and (b). Raw correlation values are in Table 1.

FIG. 3. A complex PBMC and endothelial cell co-culture system expands the coverage of biological activities relevant to inflammation. PBMC were incubated with endothelial cells and treated with SAg to activate T cell receptor-dependent responses. (a) Heat map of mean log parameter expression ratios from 3 independent experiments (n=3 replicates per experiment) showing the increase (green), decrease (red) or lack of change (black) of individual parameter levels. Color saturation reflects the magnitude of the drug effect (see scale, bottom). (b) Pairwise Pearson correlations between individual experiments, as for FIG. 2b. The dendrogram at the right shows the results of hierarchical clustering of the mean profile data (log expression ratio averaged for replicate experiments). This tree was used to determine the order of presentation of compounds in (a) and (b). Raw correlation values are in Table 2.

FIG. 4. Multi-system analysis increases detection and discrimination of compounds. 48 compounds representing 20 functional classes were tested in the 3C, SAg, and LPS systems. (a) List of drugs belonging to each class color-coded by reported mechanism of action. (b) Hierarchical clustering of active compound profiles in the individual 3C, SAg, and LPS systems. (c) Hierarchical clustering of active compound profiles from combination systems: the 3C+SAg and 3C+SAg+LPS systems. Grey compounds indicate lack of activity in a given system or combination of systems. Compounds in (b) and (c) are ordered by non-supervised hierarchical clustering of average profiles (n=3 independent experiments per drug).

FIG. 5. Drug classification by homology of function across multiple complex systems. 48 compounds were subjected to homology of function classification using the concatenated profiles from three systems (3C, SAg, and LPS). (a) Heat map of mean log parameter expression ratios from three experiments (n=3 replicates in each experiment), showing the increase (green), decrease (red) or lack of change (black) of individual parameters in each system. (b) Pearson correlations for pairwise comparisons of average profile data: positive correlations are in blue (most intense for r>0.9); black is no correlation (r˜0); and yellow indicates negative correlations. Compounds in (a) and (b) were ordered automatically by scaling and pivoting to move high correlations to the diagonal. (c) Homology of Function Map representing compound relationships visualized in two dimensions using multidimensional scaling where significant correlations (see Methods) are shown by lines. The line length is inversely proportional to the similarity of the compound profiles. Compounds are color coded by reported class as in FIG. 4.

FIG. 6 is a series of graphs depicting profiles for representative compounds (the p38 MAPK inhibitor PD169316, the CKII/NF-κB inhibitor apigenin, the HMG-CoA inhibitor atorvastatin, the steroid dexamethasone, the NF-AT inhibitor cyclosporin, the phosphodiesterase 4 inhibitor R(−)rolipram, the MEK inhibitor UO126, and the c-Raf and p38 inhibitor ZM336372) in all three systems examined (3C, SAg, LPS) at multiple doses (0.03, 0.1, 0.3, 1.0, 3.0, 10.0 μM). The highest active, but non-toxic, dose was used in all figures. Similar dose responses and toxicity studies were done on each drug examined.

FIG. 7. BioMAP parameters characterizing TH1 or TH2 cells after one or two polarizations. CD4+ peripheral blood T cells isolated by negative selection were polarized for two rounds under TH1 or TH2 conditions. A panel of markers (abscissa) of TH1 or TH2 differentiation were used to characterize the resulting T cell populations and determine the best parameters for identifying differences between TH1 and TH2 cells after each polarization. Data presented as the difference in the percentage of CD4+ T cells expressing each marker under TH1 or TH2 conditions (TH1-TH2% Positive). Data shown is mean±SD of 3 separate polarizations using different donor CD4+ cells. Differences significantly different from 0 were determined by using a Student's t test and shown as open squares.

FIG. 8. Effects of drugs on TH1 and TH2 polarization. CD4+ peripheral blood T cells isolated by negative selection were polarized for two rounds under TH1 or TH2 conditions. During the second round of polarization, drugs or solvent controls were added to the culture every other day (days 1, 3, and 5). A panel of markers (abscissa of each plot) was used to characterize the TH1 or TH2 state of the CD4+ population after the second polarization. Data has been normalized to the percentage of cells expressing the given marker under media only conditions (short dashed line). Circled data points represent normalized ratios falling outside the 95% confidence interval for the solvent control data (long dashed line). TH1 marker changes below 1 or TH2 marker changes above 1 for the TH1 polarization condition signify a shift in the population towards the TH2 state. Conversely, TH1 markers above 1 or TH2 markers below 1 for the TH2 polarization condition signify a shift of the population towards the TH1 state. Note that anti-IL-12 greatly impairs the ability of cells to polarize toward the TH1 state, but has little effect on the polarization toward the TH2 state since it is already present in the culture for TH2 polarization. Data is representative of two experiments.

FIG. 9. Effects of drugs LPS-stimulated monocyte cytokine secretion. CD14+ monocytes were enriched by adherence to 24-well plastic tissue culture dishes for 1 hr and unbound cells were removed. Monocytes were stimulated with 1 μg/ml LPS for 5 hr in the presence of 2 μM monensin (a secretion inhibitor). Drugs or solvent control (DMSO) at the indicated concentrations were added 15 minutes before LPS stimulation and were present for the entire 5 hr. Annexin V staining indicates apoptotic cells. Intracellular cytokines were detected by fixing and permeabilizing the cells after scraping them off of the culture dish. Data is presented as the percentage of cells staining positive for a particular parameter. Data is representative of three experiments performed on different days.

### DETAILED DESCRIPTION OF THE INVENTION

Methods are provided for mechanism-based drug discovery in complex primary human cell systems, which systems allow rapid and reproducible characterization of compound mechanisms of action and related activities. The systems described here have multiple applications to drug discovery. The broad coverage of biology provides a useful tool for compound validation, e.g. to determine specificity of action for a candidate agent. Multiplexed activity profiling in scalable complex cellular systems has the potential to rapidly characterize pathways and mechanisms of action of novel molecules.

The strength of the present methods derives from the complex, combinatorially-determined system responses, may be enhanced by parallel interrogation of systems in which there is a simultaneous activation of multiple signaling pathways. The methods optionally utilize a pauciparameter analysis, where relatively small numbers of parameters are read out. Combinations of multiple cell types may be used, particularly including at least one primary cell type. The readout information may be combined into multi-system analysis for enhanced resolution.

Applications of the methods of the invention may include large scale gene function screening and target validation, integration of biology and pathophysiology into target validation and drug development, improving the efficiency of drug development programs; and large scale characterization and analysis of environment- and cell differentiation-dependent biological responses.

As used herein, a “system” of the invention comprises one or more cell types, factors, and a test agent for classification analysis. In most cases, a system will further comprise samples of cells and factors, lacking the test agent, which samples serve as a control. The system may also comprise samples of cells and factors, in the presence of a known agent, which samples also serve as a control. Samples within the system may comprise different combinations of the factors. Each said sample may be present in replicate, so as to control for biological variation. After exposure to the test agent, the cellular response is measured by the evaluation of parameters. The change in parameters resulting from the presence of an agent is compared with controls and/or datasets obtained from other agents, particularly where such agents include those with known biological activities. If there is an effect in the presence of the test agent, then the target of that effect and the mechanism of action can likewise be determined. By being able to compare the effect on a family of parameters as to the degree of change in the absence of the compounds, the function of the compounds can be compared, the pathways affected identified and side effects predicted.

The results can be entered into a data processor to provide a biomap dataset. The biomap will include the parameter readouts from one or more systems. The biomap is prepared from values obtained by measuring parameters or markers of the cells in the presence and absence of different agents in a system, as well as comparing the presence of the agent of interest and at least one other state, usually the control state, which may include the state without agent or with a different agent. The parameters include cellular products or epitopes thereof, as well as functional states, whose levels vary in the presence of the factors. Desirably, the results are normalized against a standard, usually a “control value or state,” to provide a normalized data set. Values obtained from test conditions can be normalized by subtracting the control values from the test values, and dividing the corrected test value by the corrected stimulated control value. Other methods of normalization can also be used; and the logarithm or other derivative of measured values or ratio of test to stimulated or other control values may be used. Data is normalized to control data on the same cell type under control conditions, but a biomap may comprise normalized data from one, two or multiple cell types and assay conditions.

The biomap will comprise values of the levels of at least two different parameters from samples in the system, and may comprise values from at least about 3, at least about 4, and not more than about 20 parameters. The results provided herein demonstrate that small numbers of parameters (pauciparameter analysis) can be highly informative, where the number of parameters may be less than about 10, and more than about 3, usually more than about 5.

Depending on the use of the biomap, the biomap may also include the parameter values for multiple systems, where a first biomap, a second biomap, a third biomap, etc. are compared. Compilations of biomaps are developed that provide the values for a sufficient number of alternative systems to allow comparison of values. This proves to be a particularly powerful approach to increase discrimination and classification of diverse biological activities.

Mathematical systems can be used to compare biomaps, and to provide quantitative measures of similarities and differences between them. For example, the biomaps in the database can be analyzed by pattern recognition algorithms or clustering methods (e.g. hierarchical or k-means clustering, etc.) that use statistical analysis (correlation coefficients, etc.) to quantify relatedness of biomaps. These methods can be modified (by weighting, employing classification strategies, etc.) to optimize the ability of a biomap to discriminate different functional effects. Profile data, e.g. averaged, normalized, log normalized, etc. may be ordered in a correlation plot by coupling multidimensional scaling and pivoting to move high correlations toward the diagonal. Statistical analyses allow objective evaluation of the significance of all pairwise correlations between agent activities. Multidimensional scaling may be used to visualize the relationships between agents.

Multiple signaling pathways are activated by contacting the cells with factors that activate such pathways. At least one factor is present in the system, usually at least two factors, more usually at least three factors, and the system may comprise at least four factors or more. Numerous factors are known that induce pathways in cells that are responsive to the factor. For the most part, factors bind to cell surface receptors, although other receptors may be involved, such as receptors at the nuclear membrane. In addition, where a factor is able to penetrate the surface membrane, through passive or active transport or through endocytosis, the factor may bind to components of the membrane, cytosol or an organelle, e.g. nucleus.

Preferably, the factors selected in a combination are related to a physiological state of interest, e.g. pro-inflammatory response; anti-inflammatory response; angiogenesis; developmental pathways of interest; and the like. Depending on the desired biomap, these factors can include cytokines, chemokines, and other factors, e.g. growth factors, such factors include interleukins; GM-CSF, G-CSF, M-CSF, TGF, FGF, EGF, TNF-α, GH, corticotropin, melanotropin, ACTH, etc., extracellular matrix components, surface membrane proteins, such as integrins and adhesins, and other components that are expressed by the targeted cells or their surrounding milieu in vivo.

Combinations of interest include the set of factors associated with endothelial cells, e.g. EGF, FGF, VEGF, insulin, etc., cytokines, such as the interleukins, including IL-1 IL-3, IL-4, IL-8 and IL-13; interferons, including IFN-α, IFN-β, IFN-γ; chemokines; TNF-α, TGFβ, proangiogenic and anti-angiogenic factors, etc.

A chronic Th2 assay combination can be defined by the culture of responsive cells with TNF-α and/or IL-1 and IL-4 for 24 hours. Inflammation in chronic Th2 environments, such as asthma, is characterized by the presence of TNF-α, IL-1 and IL-4, but not IFN-γ.

T cell cultures may include combinations of anti-CD3+IL-2±IL4±IFN-γ±IL-12±anti-IL-4 or anti-IFN-≢5). The disease environment in psoriasis includes IL-12, IFN-γ and TNF-α. The disease environment in Crohn's disease includes IL-1, TNF-α, IL-6, IL-8, IL-12, IL-18, and IFN-γ. The disease environment in rheumatoid arthritis includes TNF-α, IL1, IL-6, IL-10, IL-15, MIP1, MCP-1, and TGFβ. The disease environment in asthma includes IL-1α, IL-4, IL-5, IL-6 and GM-CSF. Macrophages are responsive to IL-4 and other IL factors, M-CSF, and GM-CSF. Cancer cells may be used in a system to investigate immune responsiveness, neoplastic proliferation, angiogenesis; and the like, where factors of interest include chemokines; angiogenic factors; cytokines, such as IL-10; steroids, e.g. estrogen, progesterone; testosterone; anti-Her-2/neu; epidermal growth factor; FGF; IGF-I; etc.

Hematopoiesis environments may include flt-2; stem cell factor; IL-6; IL-3; IL-7; LIF; etc.

Systems for investigating pro- and anti-inflammatory systems may also include sugerantigens as a stimulus of the T cell receptor complex, or lipopolysaccharide (LPS) as a stimulator of toll receptor signaling (LPS system). Factors of interest include IL-1α; IL-1β; IL-2; IL3; IL-4; IL-5; IL-6; IL-7; IL-8; IL-9; IL-10; IL-11; IL-12; IL-13; IL-18; M-CSF; G-CSF; GM-CSF; MCP-1; MIG; IFN-α; IFN-β; IFN-γ; TGFβ; histamine; PHA, anti-CD3; anti-CD28, ConA; anti-IL-1, anti-IL-2, anti-TNF-α, anti-IFN-γ, anti-IL-12; anti-IL-4; anti-TGFβ; etc.

In one embodiment of the invention, the assay system comprises one or more primary cells. As used herein, the term “primary cell” refers to those cells present in the initial cell cultures established from a tissue, and refers to cells derived from subsequent passages, usually less than about 10 passages, and preferably less than about 5 passages. Adherent cells in primary cultures usually grow until they cover the culture dish surface, i.e. they show contact inhibition. Primary cells cannot normally be grown in culture indefinitely. Those cell lines that proliferate indefinitely in culture may be referred to as “immortal” or “immortalized cell lines”, and for the purposes of the present invention are distinct from primary cells. Some immortalized cell lines are tumorigenic, and may be referred to as “transformed” cell lines. Although such permanent cell lines have been particularly useful for many types of experiments, they are less preferred for the methods of the present invention.

Many cell types find use in the systems of the present invention. Included, without limitation, are cells involved in inflammatory responses. Such cells may include endothelial cells, e.g. primary microvasculature, HUVEC, aortic endothelial cells, etc.; blood mononuclear cells or a subset of cells derived therefrom, e.g. T cells, B cells, natural killer cells, monocytes, macrophages, etc.; blood polymorphonuclear cells or a subset of cells derived therefrom, e.g. eosinophils, basophils, neutrophils, megakaryocytes; etc., dendritic cells; thymic epithelial cells; cortical dendritic cells; etc. The component cells maybe further subdivided, e.g. T cells can be selected for Th1/Th2 polarization; CD4+; CD8+; cells in the B cell lineage may be divided into plasma cells, B cells, pre-B cells; etc.

The assay system may comprise two or more cell types, which may be primary cells, cell lines, or combinations thereof. Systems encompassing multiple cell types not only respond to perturbations of each cell type's intracellular signaling networks, but also to inhibition of pathways of communication between cells. Thus, systems comprising multiple cell types can provide additional coverage of biological function space.

Combinations of interest include, without limitation, endothelial cells and leukocytes; leukocytes and antigen presenting cells; cancer cells and endothelial cells; cancer cells, antigen presenting cells and leukocytes; mesenchymal stem cells or hematopoietic stem cells and stromal cells; thymocytes and thymic epithelial cells and/or cortical dendritic cells; neural stem cells and endothelial cells; and the like.

In the screening assays for genetic agents, polynucleotides are added to one or more of the cells in a panel in order to alter the genetic composition of the cell. The output parameters are monitored to determine whether there is a change in phenotype affecting particular pathways. In this way, genetic sequences are identified that encode or affect expression of proteins in pathways of interest, particularly pathways associated with aberrant physiological states.

Assay combinations, usually employing cell cultures, are provided that simulate physiological cell states of interest, particularly physiological cell states in vivo, usually using the same type of cells or combinations of cells. These cell cultures are created by the addition of a sufficient number of different factors to provoke a response that simulates cellular physiology of the state of interest and to allow for the status of cells in culture to be determined in relation to a change in an environment. The state of interest will normally involve a plurality of pathways where the pathways regulate a plurality of parameters or markers identifying a phenotype associated with the state of interest.

The phenotype can be generated by including a plurality of factors that induce pathways affecting the production of the phenotype by the up or down regulation of formation of the parameters as detectable products or may be based on the nature of the cell, e.g. neoplastic primary cells, cell lines, etc., where the factors enhance the response of the cells in vitro to more closely approximate the response of interest. The factors are naturally occurring compounds, e.g. known compounds that have surface membrane receptors and induce a cellular signal that results in a modified phenotype, or synthetic compounds that mimic the naturally occurring factors. In some instances, the factors will act intracellularly by passing through the cell surface membrane and entering the cytosol with binding to components in the cytosol, nucleus or other organelle. In providing the environment by use of the factors or mimetics, one provides the activities of the factors to the environment, using the naturally occurring factors or their mimetics. In referring to factors, it is understood that it is the activities of the factors that are of interest and not necessarily a particular naturally occurring factor itself.

The nature and number of parameters measured generally reflects the response of a plurality of pathways. The subject approach provides for robust results having enhanced predictability in relation to the physiological state of interest. The results may be compared to the basal condition and/or the condition in the presence of one or more of the factors, particularly in comparison to all of the factors used in the presence and absence of agent. The effects of different environments are conveniently provided in biomaps, where the results can be mathematically compared.

For screening assays with genetic agents, the same approach will be used as above. The genetic agents are added to cells, which are placed in a medium where one or more factors may be present to provide a desired environment, namely an environment of interest, such as a physiological environment involved with an aberrant, e.g. diseased, state. Parameters associated with the pathways related to the physiological state are monitored. Where the parameters show a pattern indicating the up or down regulation of a pathway, the genetic agent is deduced to encode or affect the expression of a member of the pathway. In this way one can determine the role a gene plays in the physiological state of interest, as well as define targets for therapeutic application.

Once biomaps have been prepared for pathways and/or environments of interest, assays may be carried out with or without the factors. Knowing the variation in parameters with individual factors and different combinations of factors, one can compare the effect of an agent on a cell culture by measuring parameters that have been previously measured in different assay combinations. The observed effect of the agent on the levels of the different parameters may then be correlated with the observed effect of the factors and combinations of factors in the biomap dataset.

In referring to simulation to a physiological state, the simulation will usually include at least three different regulated features (parameters) shared with in vivo cell counterparts in normal or diseased states. Alternatively, the simulation may include a cell culture system that allows discrimination of modifications in at least three different signaling pathways or cell functions operative in vivo under conditions of interest.

The results can be entered into a data processor to provide a biomap dataset. Algorithms are used for the comparison and analysis of biomaps obtained under different conditions. The effect of factors and agents is read out by determining changes in multiple parameters in the biomap. The biomap will include the results from assay combinations with the agent(s), and may also include one or more of the control state, the simulated state, and the results from other assay combinations using other agents or performed under other conditions. For rapid and easy comparisons, the results may be presented visually in a graph of a biomap, and can include numbers, graphs, color representations, etc.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof.

Preferred parameters are informative, that is they have a robust modulation in response to one or more individual factors or agents of the system; and additionally may have potential or known relevance to the system, e.g. inflammation, cancer biology, etc. As previously discussed, the set of parameters selected is sufficiently large to allow distinction between agents, while sufficiently selective to fulfill computational requirements.

A parameter may be defined by a specific monoclonal antibody or a ligand or receptor binding determinant. Parameters may include the presence of cell surface molecules such as CD antigens (CD1-CD247), cell adhesion molecules including α₄β₇ and other integrins, selectin ligands, such as CLA and Sialyl Lewis x, and extracellular matrix components. Parameters may also include the presence of secreted products such as lymphokines, including IL-2, IL-4, IL-6, growth factors, etc.

For T cells these parameters may include IL-1R, IL-2R, IL4R, IL-12Rβ, CD45RO, CD49E, tissue selective adhesion molecules, homing receptors, chemokine receptors, CD26, CD27, CD30 and other activation antigens. Additional parameters that are modulated during activation include MHC class II; functional activation of integrins due to clustering and/or conformational changes; T cell proliferation and cytokine production, including chemokine production. Of particular importance is the regulation of patterns of cytokine production, the best-characterized example being the production of IL-4 by Th2 cells, and interferon-γ by Th1 T cells. For endothelial cells, parameters include ICAM-1, E-selectin, IL-8, HLA-DR, VCAM1, GRO-α, ENA-78, etc.

Other parameters of interest include, without limitation, MIG (CLCX9); IP-10; Eotaxin-1; Eotaxin-3; MCP-1; RANTES; Tarc; CD31; alphavbeta3; P-selectin (CD62P); CD34; CD14; CD40; CD38; CD55; CD69; CXCR2; CD95; fibronectin; HLA-ABC; GROalpha; MCP4; TAPA-1; integrin alphaVbeta5; E-Cadherin; CD44; von Willebrand factor; CD3; CD25; CD141; CD142; CD143; CD151; MCP-1; cutaneous lymphocyte antigen (CLA); CXCR3; CCR3; TNF-α; IFN-γ; IL-2; IL-4; IL-1alpha; M-CSF; integrin alpha4beta7; integrin alphaEbeta7; L-selectin; EGF-R; HLA-DR (CD74); CD44; carcinoembryonic antigen (CEA, CD66e); integrin α₅β₁; HLA-I; poly-Ig-receptor; CA-19-9; CD95; integrin α₂β₁; integrin α₃β₁; integrin α₆β₁; integrin α₆β₄; integrin αᵥ; laminin 5; urokinase-type plasminogen activator receptor (uPAR); TNFR-I; lactate dehydrogenase (LDH); mitochondrial cytochrome c; APO2.7 epitope; active caspase-3; Ki-67; and PCNA.

Agents of interest include drugs and genes, which induce characteristic signature profiles. Those of skill in the art will appreciate that, while the invention is illustrated with a number of important genes and drugs relating to inflammation and its control, the invention can be applied to any gene or drug. The completion of the human genome has made available the full complement of human genes and, in combination with modern approaches to chemical diversity, has opened up unparalleled opportunities for advances in biology and medicine.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Agents are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; encode a dominant negative mutation, or dominant or constitutively active mutations of native sequences; altered regulatory sequences, etc.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746.

cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

### Materials and Methods

This Example provides information regarding the materials and methods used throughout the remaining examples to illustrate the invention and the advantages provided thereby. Those of skill in the art will recognize that a variety of materials different from those exemplified herein can be used to practice the invention and that variation of the illustrated methods is within the routine skill in view of the teachings herein.

Methods

Cytokines, antibodies, and reagents. Recombinant human IFN-γ, TNF-α, and IL-1β were from R&D Systems (Minneapolis, Minn.). Murine IgG was from Sigma (St. Louis Mo.). Mouse anti-human tissue factor (mIgG₁) was from CALBIOCHEM (San Diego, Calif.). Mouse anti-human ICAM-1 (mIgG₁) was from Beckman Coulter (Fullerton, Calif.) and mouse anti-human E-selectin (mIgG₁) was from HyCult Biotechnology (Uden, The Netherlands). Unconjugated mouse antibodies against human VCAM-1 (mIgG₁), CD31 (mIgG₁), HLA-DR (mIgG₂ₐ), CD3 (mIgG₁), CD40 (mIgG₁), CD69 (mIgG₁), MIG (mIgG₁), MCP-1 (mIgG₁), CD14 (mIgG₁), IL-1α (mIgG₁), and CD38 (mIgG₁) were obtained from BD Biosciences (San Jose, Calif.). Unconjugated mouse antibodies against IL-8 (mIgG₁) and M-CSF (mIgG₁) were obtained from R&D Systems. Polyclonal goat antibodies against TNF-α, IFN-γ, and IL-1β and control goat IgG were obtained from R&D Systems.

Apigenin, UO126, budesonide, dexamethasone, genistein, zearalenone, β-zearalenol, azathioprine, prednisolone, leflunomide, nabumetone, AA861, and cyclosporin A were obtained from Sigma. PD098059, PD169316, SKF-86002, SB220025, mevastatin, nordihydroguaiaritic acid (NGDA), FK-506, and rapamycin were from Calbiochem (San Diego, Calif.). Atorvastatin and simvastatin were from LKT Laboratories (St. Paul, Minn.). Recombinant human IL-1ra, and IL-10 were from R&D Systems. Ro-20-1274, R(−)rolipram, DRB, PP2, and PP1 were from BIOMOL (Plymouth Meeting, Pa.). Mycophenolic acid, WHI-P131, ZM39923, wortmannin, SC-560, NS-398, LM1685, AG490, AG126, SC68376, and SB239063 were from Calbiochem. ZM336372, radicicol, 17-AAG, SP600125, lovastatin, LY294002, FR122047, DUP697, and geldanamycin were from Tocris (Ellisville, Mo.).

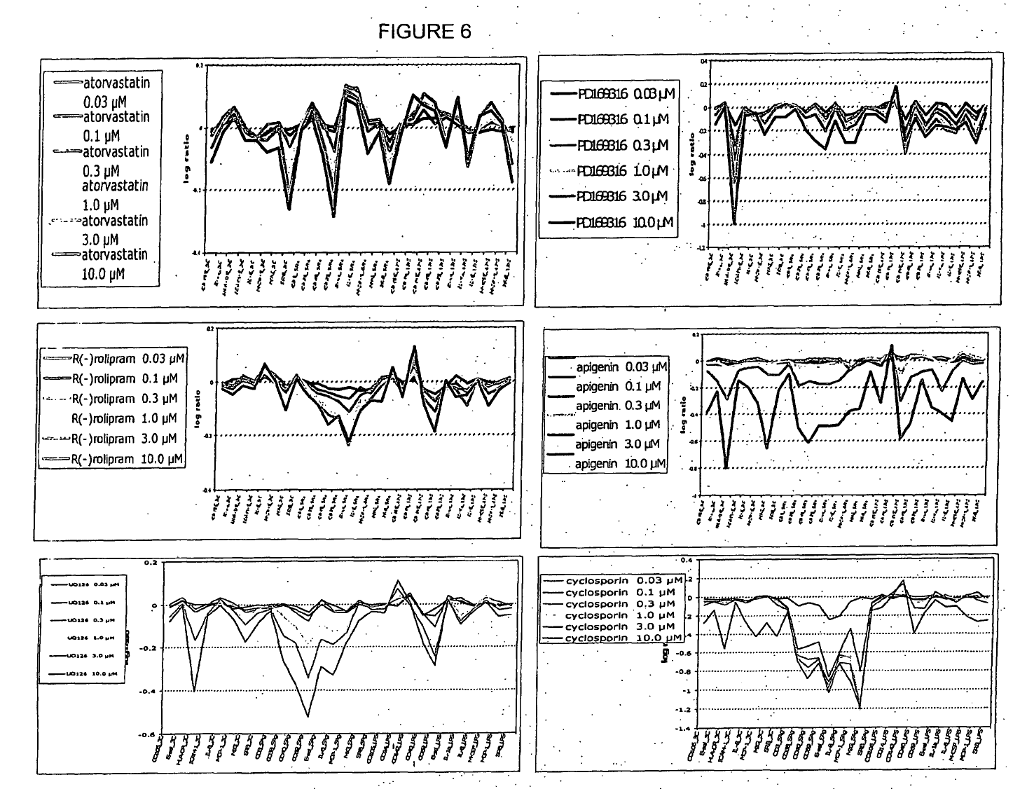

Compounds were evaluated over a range of concentrations (for example, see FIG. 6) and data shown are at concentrations that do not result in cell toxicity. Staphylococcal enterotoxin B, toxic shock syndrome toxin-1 (Staphylococcal enterotoxin F) from S. aureus and lipopolysaccharide from Salmonella enteritidis were obtained from Sigma.

Cell culture. Human umbilical vein endothelial cells (HUVEC) were obtained from Cascade Biologics (Portland, Oreg.) and cultured in EGM-2 medium containing supplements provided by the manufacturer and 2% heat inactivated fetal bovine serum (Hyclone, Logan, Utah) and subcultured with 0.05% trypsin-0.53 mM EDTA (Mediatech, Herndon, Va.) as described by the manufacturer. Peripheral blood mononuclear cells (PBMC) were prepared from buffy coats (Stanford Blood Bank, Stanford, Calif.) by centrifugation over Hisopaque-1077 (Sigma). Experiments were performed by culturing HUVEC in microtiter plates (Falcon; BD Biosciences), in the presence of cytokines (IL-1β, 1 ng/ml; TNF-α, 5 ng/ml; and IFN-γ, 100 ng/ml), activators (SAg, 20 ng/ml or LPS, 0.2 ng/ml), and/or PBMC (7.5×10⁴) for the indicated times. Drugs were added 1 hr before stimulation and were present during the whole 24 hr stimulation period.

Cell-based ELISAs. Cell-based ELISAs were carried out essentially as described by Melrose et al. (1998) J Immunol 161:2457-64. Briefly, microtiter plates containing treated and stimulated HUVEC (or HUVEC/PBMC) were blocked, and then incubated with primary antibodies or isotype control antibodies (0.01-0.5 μg/ml) for 1 hr. After washing, plates were then incubated with a peroxidase-conjugated anti-mouse IgG (Promega) secondary antibody or a biotin-conjugated anti-mouse IgG antibody (Jackson ImmunoResearch, West Grove, Pa.) for 1 hr followed by streptavidin-HRP (Jackson ImmunoResearch) for 30 min. Plates were washed and developed with TMB substrate (Clinical Science Products, Mansfield, Mass.) and the absorbance (OD) was read at 450 nm (subtracting the background absorbance at 650 nm) with a Molecular Devices SpectraMAX 190 plate reader (Molecular Devices, Sunnyvale, Calif.).

siRNA transfection. Early passage (<5) exponentially growing HUVEC cells were harvested, washed once with PBS, and resuspended at 2×10⁶ cells in 100 μl Nucelofection solution (Human Umbilical Vein Endothelial Cell Nucleofector Kit, AMAXA, Koeln, Germany). TNFRI siRNA (SEQ ID NO:1 MGTGCCACAAAGGMCCTAC; 15 μl of a 20 μM solution; Dharmacon, Lafayette, Colo.) was added to the cell suspension, transferred into an electroporation cuvette, and electroporated using the U-1 setting. The cell suspension was then transferred into a separate tube containing 3 ml EGM-2 media (Clonetics), incubated at 37° C. for 10 minutes, and plated into microtiter plates (25,000 cells/well) for cytokine activation and ELISA analysis as described above.

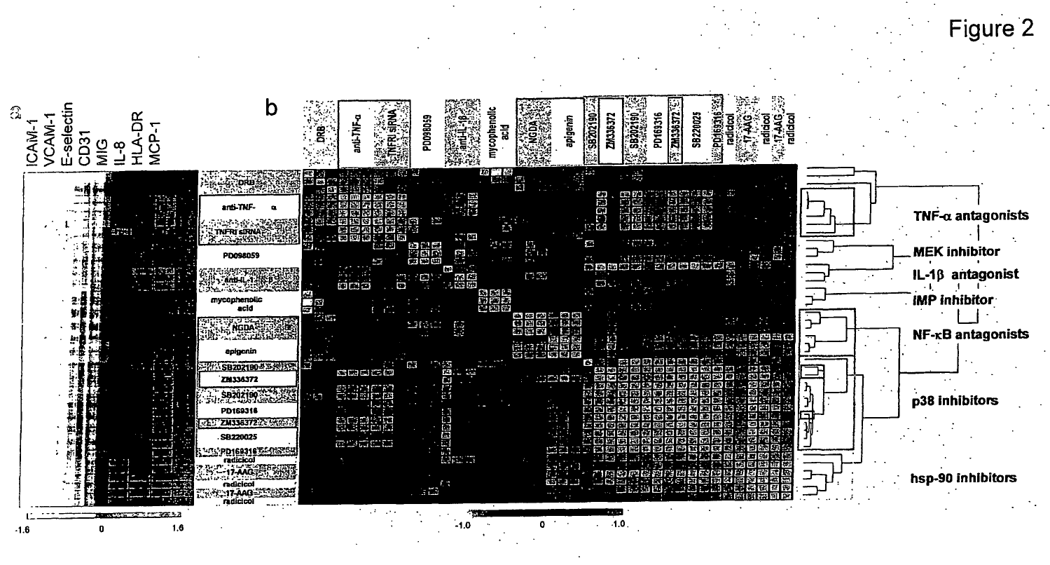

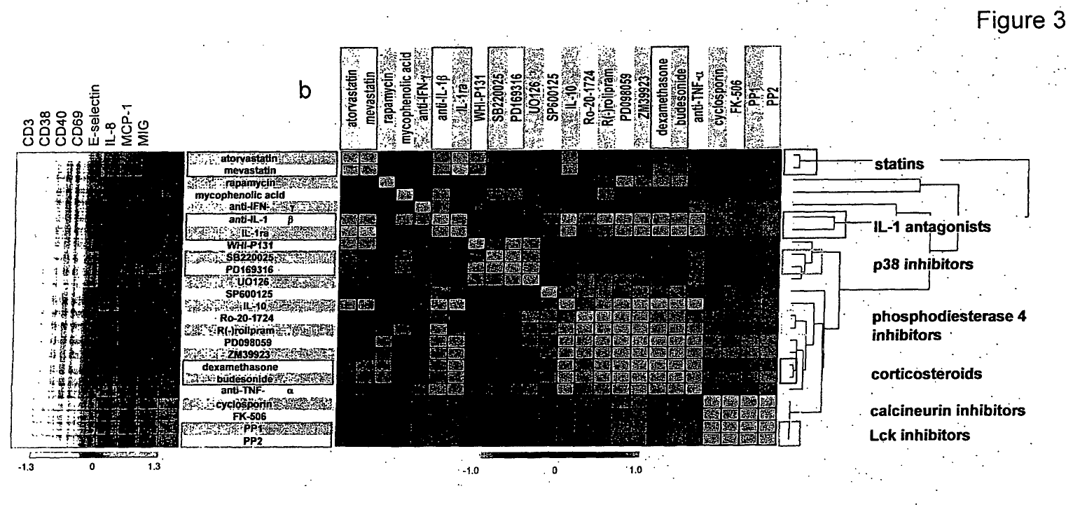

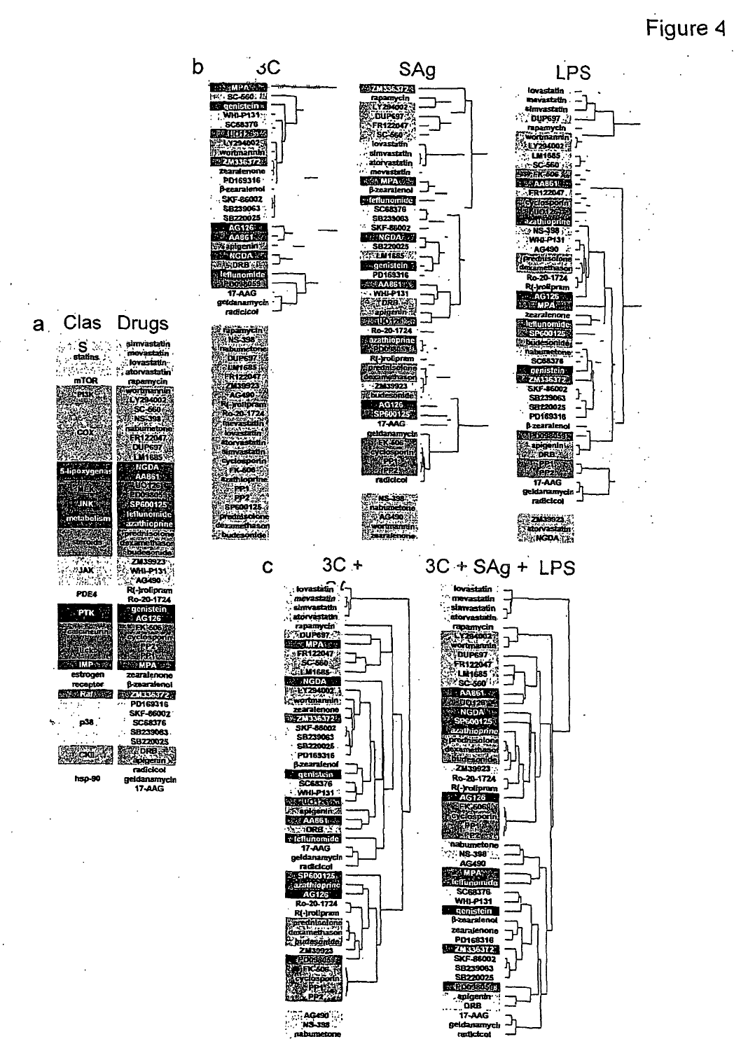

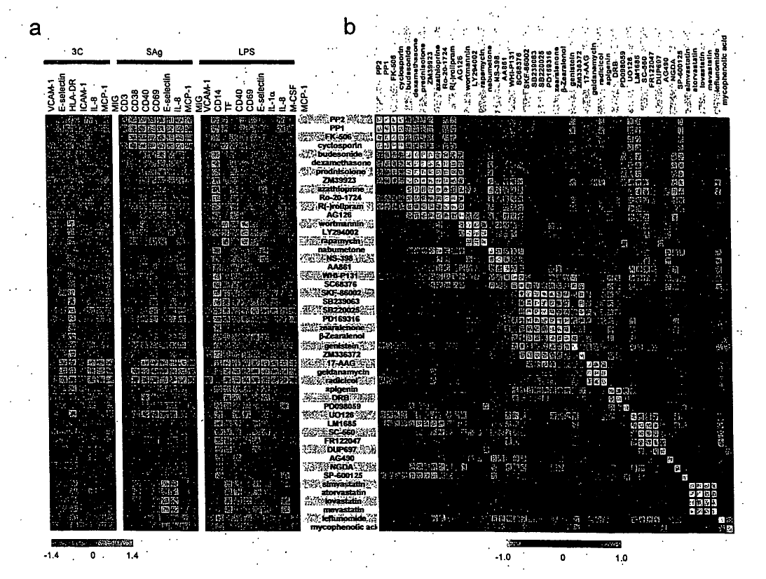

Data analysis. Mean OD values for each parameter were calculated from triplicate samples per experiment. Mean values were then used to generate ratios between treated (e.g. drug or siRNA) and matched control (e.g. media or DMSO) parameter values within each experiment. These normalized parameter ratios were then log₁₀ transformed. Log expression ratios were used in all Pearson correlation calculations (Partek Pro, version 5.1; Partek, St. Charles, Mo.). 66% confidence intervals for Pearson correlations were determined by bootstrap resampling (Efron & Tibshirani, An Introduction to the Bootstrap, (Chapman and Hall, New York, 1993). Individual experiment profiles (FIG. 2) or averaged profiles (n=3 experiments per drug; FIGS. 3 and 4) were ordered by hierarchical clustering (Pearson correlation metric with average distance).

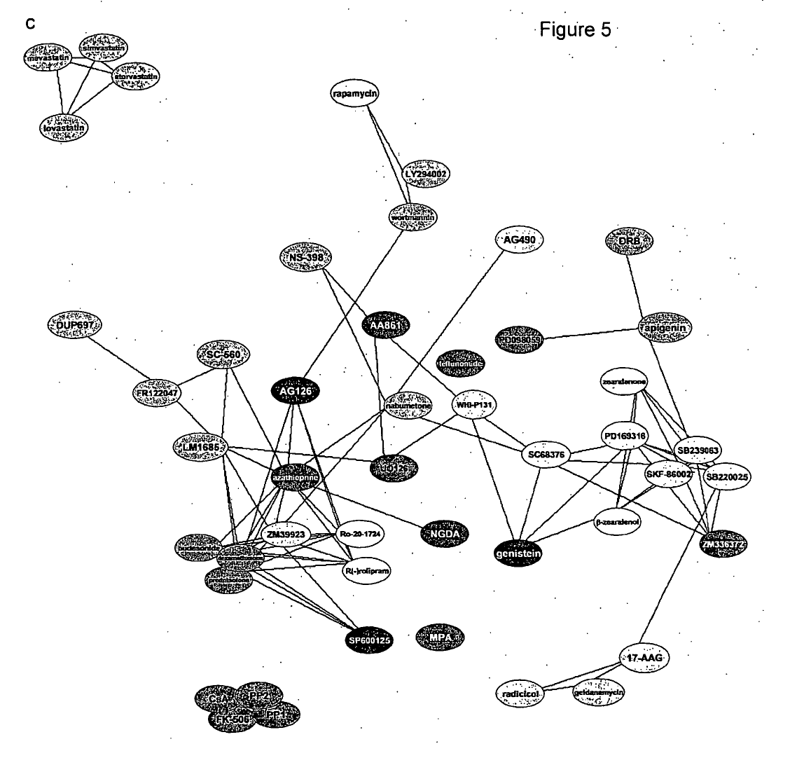

Averaged profile data in FIG. 5 were ordered in the correlation plot by coupling multidimenstional scaling and pivoting to move high correlations toward the diagonal. Significant correlations were determined by 1) creating a distribution of Pearson correlations using randomized data made from permuting the empirical profiles, 2) selecting a Pearson correlation (0.67 in FIG. 5) to minimize the FDR (Tusher et al. (2001) Proc Natl Acad Sci USA 98:5116-21). (2% in FIG. 5), or the ratio of the number of correlations greater than this selected Pearson correlation in the randomized data to the number of correlations greater than this selected Pearson correlation in the empirical data, and 3) applying this cut-off Pearson correlation value to the correlations between experimental profiles.

In other words, for a 2% FDR, 98% of the correlations derived from the experimental profiles are a result of a true biological effect, and not random chance. Correlations were visualized in two dimensions by multidimensional scaling using AT&T GraphViz software. Distances between compounds are representative of their similarities and lines are drawn between compounds whose profiles are similar at a level not due to chance (as defined above).

### EXAMPLE 2

### Regulators of Endothelial Cell Function

This Example illustrates the application of the present invention to the screening of compounds for altering immune and/or inflammatory conditions that involve endothelial cells. Endothelial cells were cultured as described in Example 1.

A complex, cytokine-stimulated endothelial cell inflammation system discriminates inhibitors of multiple signaling pathways. Endothelial cells modulate inflammatory responses by regulating leukocyte traffic through their expression of adhesion receptors and chemokines. In chronically inflamed tissues, endothelial cells are exposed to multiple proinflammatory cytokines including IL-1β, TNF-α, and IFN-γ. Therefore, primary human endothelial cells (EC) were stimulated with this combination of three pro-inflammatory cytokines (3C system) to simultaneously activate critical pathways and pathway interactions relevant to chronic inflammatory processes. Readouts were selected for their robust modulation in response to one or more individual cytokines or cytokine combinations or to specific drug effects (see below), and for their potential or known relevance to inflammatory biology.

From a large number of potential readouts surveyed, the following readout parameters were selected: VCAM-1, ICAM-1 and E-selectin (vascular adhesion molecules for leukocytes), MHC class II (antigen presentation), MIG/CXCL9, MCP-1/CCL2 and IL-8/CXCL8 (chemokines that mediate selective leukocyte recruitment from the blood), and CD31 (leukocyte transmigration). Proteins or bioactive molecules were measured (instead of expressed genes) because 1) unlike genes, biologically active proteins are the proximate mediators of physiologic and pathophysiologic processes; and 2) these species are readily measured in scalable high throughput assay formats. Drugs were included during the 24 hr cytokine activation period, and readout parameters were measured by ELISA. Relative changes of readout parameter expression levels in response to drug treatment in each system are presented.

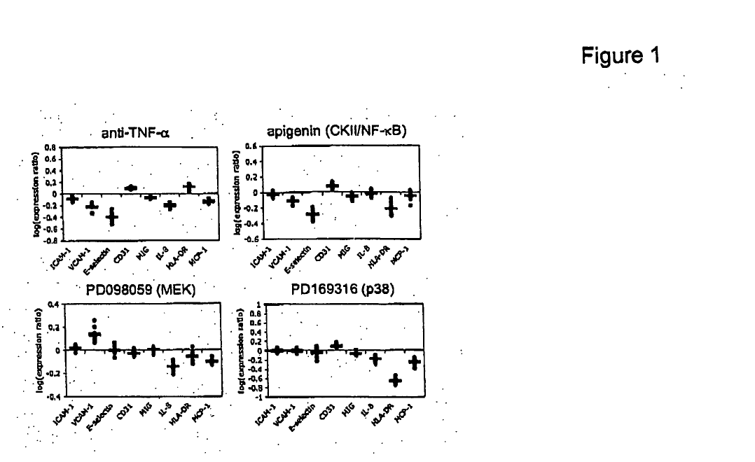

As illustrated in FIG. 1 (ten independent experiments) the 3C system responds to inhibitors of multiple different pathways and mechanisms including an antibody inhibitor of TNF-α, and the drugs apigenin (a casein kinase II inhibitor that blocks NF-κB function), PD169316 (a p38 MAPK inhibitor), and PD098059 (a MEK inhibitor). Each of these agents induces a characteristic response profile, indicating that the 3C system responds in a unique and reproducible fashion to perturbation of these signaling components. Additional dose response data are provided in FIG. 6.

### EXAMPLE 3

### Correlation of System Responses With Drug Mechanism of Action and Pathway Inhibition

To determine if system responses to drugs correlate with mechanism of action or pathway activities, we evaluated the responses of the 3C system with chemically diverse inhibitors acting on common cellular pathways. In FIG. 2a, drug activity profiles from three independent experiments per drug are presented in heat map form (green indicates that the level of the readout parameter indicated is increased relative to the no drug control, black indicates no change, and red indicates a decrease). FIG. 2b shows the results of pairwise comparison of drug activity profiles (Pearson correlation coefficient, r). Pairs of profiles with high positive correlation coefficients (most intense for values >0.9) are light blue. The order of compounds in FIG. 2a and 2b was determined by non-supervised hierarchical clustering (see tree to the right of FIG. 2b using Pearson correlation as the clustering metric).

Drugs with a common target induce homologous profiles. During inflammation, cytokine signaling leads to phosphorylation and translocation of the p38 mitogen activated protein kinase (MAPK) into the nucleus, where it participates in the transcriptional regulation of cell surface receptors and cytokines. Direct comparison of system responses to chemically distinct inhibitors of p38 MAPK reveals strong homology of function (e.g. the pairwise Pearson correlation between the mean log parameter expression ratios for PD169316 versus SB220025 in FIG. 2b is r=0.96; 66% confidence interval of [0.90, 0.99]). This functional correlation is reproducible, and extends to other p38 MAPK inhibitors as well, as indicated by the similarity of individual experimental profiles (in FIG. 2a) and by the uniformly high correlation of system responses of PD169316 and other p38 MAPK inhibitors (SB220025 and SB202190) across multiple individual experiments (see high pairwise Pearson correlations between individual PD169316, SB202190, and SB202190 experiments in FIG. 2b). In contrast, as illustrated for individual experiments in the correlation map in FIG. 2b, little or no similarity is seen in system responses to p38 inhibition versus MEK inhibition (by PD098059) or NF-κB inhibition (by apigenin, NGDA, or TNF-α antagonists) (r<0.4).

Homology of function in complex cell systems can reveal mechanistic overlap of compound activities. Two inhibitors of the molecular chaperone hsp-90, 17-MG and radicicol exhibit reproducible functional correlation (FIG. 2a and 2b) and interestingly, also exhibit functional similarity to p38 inhibitors (mean log expression ratio r=0.84, [0.78, 0.92] for 17-MG versus PD169316). In addition to direct inhibition of NF-κB activation, blockade of hsp-90 with radicicol can inhibit signaling through the p38 pathway, thus the homology of function identified here is consistent with mechanistic overlap between these inhibitors. Apigenin and nordihydroguaiaretic acid (NDGA) also exhibit function homology in the 3C system (FIGS. 2a and 2b; r=0.79, [0.69, 0.92]). Although these drugs have distinct mechanisms of action (the flavenoid apigenin has been characterized as an inhibitor of casein kinase II whereas NDGA is an inhibitor of 5-lipoxygenase) both inhibit responses mediated by the NF-κB pathway a key pathway in inflammation.

Homologous functional responses can also reveal off-target activities. The profile obtained for ZM336372, initially selected as an inhibitor of c-Raf and thus expected to inhibit the Ras/Raf/MEK/ERK pathway, showed significant functional similarity to p38 inhibitors (FIG. 2, blue boxes with p38 inhibitor cluster; r=0.75, [0.43, 0.97] versus PD169316). Consistent with these results, ZM336372 is now known to inhibit p38 MAPK in addition to c-Raf. Thus, reproducible responses in this 3C system correlate with known mechanisms of action, and can reveal off-target activities as well.

Complex cell systems are also responsive to protein and gene manipulations. Inhibition of TNF-α with an anti-TNF-α antibody (or a soluble form of the TNF receptor 1) yields a response that is highly correlated to the response obtained by knockdown of TNF receptor 1 using siRNA (FIG. 2) (r=0.92 [0.88, 0.98]). Gene knockdown in such complex cell systems may thus be useful to dissect gene function, and to predict the effects of drugs against the gene target.

These data demonstrate that signature drug response profiles not only detect, but also discriminate and allow classification of drug effects based on homology of function in this complex EC inflammation system. However, this single system, although encompassing sensitivity to multiple targets and pathways in inflammation, does not detect all classes of immunomodulatory agents. Inhibitors of immune cytokines not added to the system, for example, or immune modulators specific for leukocyte signaling pathways (e.g. T cell receptor signaling), had little or no effect (at levels not yielding toxicity) on the multiply stimulated endothelial cell system.

### EXAMPLE 4

### Multicellular Complex Systems for Enhanced Coverage of Biological and Pharmacologic Activity Space

Incorporating additional cell types can enhance the breadth of signaling pathways and inflammatory mechanisms assayable in complex systems. Therefore, a multicellular system comprising peripheral blood mononuclear cells (PBMC; a mixture of CD4⁺ and CD8⁺ T cells, monocytes, NK cells, and other mononuclear leukocytes) and EC, was evaluated using superantigen (SAg) as a stimulus of the T cell receptor complex (SAg system), or lipopolysaccharide (LPS) as a stimulator of toll receptor signaling (LPS system). Parameters selected include CD3 (T cell marker), CD14 (a monocyte marker), CD38, and CD69 (early activation markers), CD40 (a TNFR family member important for lymphocyte co-stimulation), E-selectin and VCAM-1 (endothelial adhesion molecules), tissue factor (TF, CD142; a initiator of coagulation), IL-1α and M-CSF (cytokines), and IL-8, MCP-1, and MIG (chemokines that control leukocyte recruitment). In these multicellular systems, cells respond directly to the initiating stimuli and/or to each other, resulting in a complex cascade of events.

The complex SAg system responds robustly and reproducibly to a number of compounds that are inactive or only weakly active in the endothelial inflammation system (FIG. 3). For example, FK-506 and cyclosporin A, inhibitors of calcineurin-mediated T cell receptor signaling, are potent inhibitors of the SAg system response (FIG. 3a; notice decrease (red) in multiple leukocyte and endothelial parameters). Moreover, these drugs show strong homology of function (FIG. 3b). Other compounds active in the SAg system (but not the 3C system) include IL-10, the phosphodiesterase 4 inhibitors Ro-20-1274 and rolipram, the immunosuppressant rapamycin, the JAK inhibitors WHI-P131 and ZM39923, HMG-CoA reductase inhibitors, corticosteroids, and the src-family kinase inhibitors PP1 and PP2 (FIGS. 3a and 3b). Most compounds active in the endothelial inflammation system (FIG. 2) retain activity in the more complex multicellular system (e.g. the p38 inhibitors PD169316 and SB220025 and anti-TNF-α; see also FIG. 4). Again, the order of compounds in FIG. 3 was determined by non-supervised hierarchical clustering of average profile data (n=3 independent experiments; see tree to the right of FIG. 3b). Other markers useful in this system include VCAM-1 and eotaxin-3.

Systems encompassing multiple cell types not only respond to perturbations of each cell type's intracellular signaling networks, but also to inhibition of pathways of communication between cells. Indeed, the SAg system detects inhibition of TNF-α (anti-TNF-α), IL-1β (anti-IL-1β and IL-1 receptor antagonist), and IFN-γ (anti-IFN-γ) produced endogenously by the SAg-stimulated leukocytes in the combined cell system (FIG. 3). Thus, systems comprising multiple cell types can provide additional coverage of biological function space.

### EXAMPLE 5

### Multi-System Analysis Provides Both Increased Detection and Discrimination of Compound Mechanisms

An additional approach to encompass a broad range of biology into response profiles is to combine data from multiple complex systems assayed in parallel. This proves to be a particularly powerful approach to increase discrimination and classification of diverse biological activities (FIG. 4). We chose 48 compounds from ˜20 functional classes (FIG. 4a) and evaluated them in three complex systems: the 3C system (see FIGS. 1 and 2), the SAg system (see FIG. 3), and the LPS system (see FIG. 5a). The order of compound presentation in FIG. 4 was determined by hierarchical clustering of profiles (FIG. 4b) or combinations of profiles (FIG. 4c) and differs depending on the combinations of systems used.

Evaluation of compounds in the 3C system alone is sufficient to detect (objectively defined as at least an 20% change in one parameter; a log ratio of ±0.1) 25 of the 48 compounds from 13 of the 20 compound classes (FIG. 4b, left panel; light grey compounds were inactive) and allows classification of a small subset of active compounds, including p38 inhibitors, hsp-90 inhibitors, and P13K inhibitors (as demonstrated by the hierarchical tree to the left of the compound list). Compound analysis in the SAg (FIG. 4b, middle panel) or LPS (FIG. 4b, right panel) systems increases the number of detected compounds to 43 and 45 respectively, and either of these multi-cellular systems is able to detect at least one member of all compound classes tested. However, neither the SAg nor LPS systems alone give very effective discrimination and classification of active compounds (see dendrograms to the right of the compound lists in FIG. 4b). For example, the hsp-90 inhibitors radicicol and geldanamycin are not differentiated from the calcineurin (FK-506 and cyclosporine) and src-family inhibitors (PP1 and PP2) in the SAg system.

Combining profiles from individual systems into “multi-system profiles” (FIG. 4c) dramatically improves the quality of the compound classification by homology of function. In the combined 3C and SAg system, HMG-CoA inhibitors, calcineurin inhibitors, src-family inhibitors, steroids, phosphodiesterase 4 inhibitors, and Cox inhibitors all form discrete clusters, while p38, hsp-90 and PI3K inhibitor clusters (from the 3C system) are maintained. The addition of the LPS system profiles in series with the 3C and SAg systems (FIG. 4c, right panel) allows optimal detection of the 48 compounds tested and further improves the discrimination of functional classes.

Although hierarchical clustering as in FIG. 4 illustrates the power of multi-system profiling to classify compounds by homology of function, this method may obscure significant similarities between compounds that are on different branches of the tree. Therefore, we applied additional statistical analyses (see Methods) to the compound profiles in all three systems to allow objective evaluation of the significance of all pairwise correlations between compound activities (FIG. 5). Profile data from all three systems were first concatenated into 25 parameter profiles (FIG. 5a), and were compared to each other by pairwise Pearson correlation (FIG. 5b; the intensity of blue indicates the degree of positive correlation). Multidimensional scaling was used to visualize the relationships between compounds (FIG. 5c): in this graph, distance between compounds is scaled to reflect their degree of similarity and lines are used to connect drugs whose multi-system profiles show statistically significant similarity.

Examination of the resulting Homology of Function Map (FIG. 5c) demonstrates that the majority of compound classes known to share a common mechanism (color coded as in FIG. 4) are significantly related to each other. Interestingly, compounds with less target specificity, such as the general tyrosine kinase inhibitors AG126 and genistein or the JAK inhibitors ZM39923, WHI-P131, and AG490 show no significant functional similarity with each other and instead show similarity to compounds from divergent classes, reflecting the unique biological consequences of their inhibition of multiple molecular targets. The two designated 5-lipoxygenase inhibitors AA861 and NGDA also yield distinctive biological responses, reflecting potential off-target activities of NGDA. Indeed, AA861 was a rationally designed inhibitor selected for enhanced 5-lipoxygenase activity and fewer side-effects¹⁵. Interestingly, while NDGA shows homology of function to the CKII inhibitor apigenin in the 3C system due to the ability of both compounds to inhibit NF-κB-dependent signaling (FIG. 2), multi-system analysis allows them to be discriminated. DRB, another CKII inhibitor, shows significant homology of function to apigenin, although these two compounds did not cluster together in the 3C system alone (see FIG. 2).

In some cases, multi-system analysis reveals similarity of functional responses induced by mechanistically distinct drugs. The observation that the mTOR antagonist rapamycin exhibits high homology of function to the general PI3K inhibitors LY294002 and wortmannin is consistent with the known regulation of p70S6K (an mTOR target) by PI3K. The phosphodiesterase 4 inhibitors (Ro-20-1724 and rolipram) cluster with corticosteroids (dexamethasone, budesonide, and prednisolone) consistent with the similarity of their effects on leukocyte signaling in the SAg and LPS systems. Interestingly, the non-steroidal fungal estrogen receptor agonists zearalenone and β-zearalenol both clustered with a large group of p38 MAPK inhibitors, an effect potentially related to the reported ability of estrogen to modulate p38 signaling.

At the same time, careful examination of compounds with correlated functions demonstrates that they are distinguishable in particular systems. For example, the phosphodiesterase inhibitors decrease M-CSF expression in the LPS system (in contrast to corticosteroids) and rapamycin distinguishes itself from either PI3K inhibitor by upregulating CD69 and E-selectin in the SAg system (FIG. 5a). Thus, examination of compound profiles in multiple systems allows both discrimination and identification of mechanistic overlap. Additional discriminatory power among inhibitors of these related biological responses could be obtained by adding additional complex systems.

In addition, one may include in the database reference BioMaps generated from assay panels containing cells with genetic constructs that selectively target or modulate specific cellular pathways (e.g. NFAT, calcineurin, NFOB, MAP kinase, and the like), or cells that contain known genetic mutations, e.g. Jurkat cell lines that lack Lck, CD45, or the like (see Yamasaki, 1997, J. Biol. Chem. 272:14787).

The ability to inhibit cellular responses to proinflammatory cytokines is a common feature of many anti-inflammatory compounds, and serves as the basis of anti-inflammatory cell-based screens in drug discovery. For example, many anti-inflammatory compounds including corticosteroids, immunosuppressants, proteosome inhibitors, various kinase inhibitors, and others have been shown to inhibit endothelial cell responses induced by IL-1β or TNF-α. Such assays detect but do not effectively discriminate or classify compounds with different mechanisms of action. The methods of the present invention provide more discrimination between compounds with different mechanisms of action, through a set of human cell-based model systems that incorporate increased levels of complexity with relevance to inflammatory disease biology. Such systems are useful for the rapid identification of effective new therapeutics. In testing the performance of these systems with known pharmacologic agents, it was discovered that the responses measured in these complex systems were surprisingly robust and reproducible, and could be employed for efficient classification of compounds according to their functional activities.

One aspect of the current study is that relatively small sets of parameters can provide extensive coverage of biological space relevant to cell and tissue level inflammation. This broad sensitivity may be an innate property of complex cellular systems, in which the level of each receptor or cytokine parameter measured is an indirect reflection of pathway interactions mediated by hundreds of signaling components. Functional discrimination depends on the empirical selection of systems and parameters that provide maximum information content. Moreover, because biological functions are context dependent, analysis in several complex systems in parallel dramatically enhances the breadth of functional responses that can be detected and distinguished. In addition to the drugs discussed, these model systems detect known and novel gene components of the NF-κB, PI3K/Akt, Ras/ERK, and IFN-γ pathways, allowing efficient and automated prediction of gene functional networks.

### EXAMPLE 6

### Regulators of T Cell Differentiation and Polarization

This Example illustrates how the methods of the present invention can be used to identify or characterize regulators of T cell mediated inflammation and immunity, such as regulators of the TH1/TH2 polarization process. A set of assay combinations that reproduces aspects of the differentiation and polarization response of adult T cells is provided.

Adult human peripheral blood CD4+ T cells are used in this illustrative embodiment of the invention. Other cells that can be used include adult peripheral blood CD8+ T cells, isolated populations of CD4+ or CD8+ T cells, and CD4+ or CD8+ memory or naive T cells. Peripheral blood mononuclear cells are isolated from blood by Ficoll-hypaque density gradient centrifugation as described (see Ponath, 1996, JEM 183:2437). CD4+ T cells are then isolated by negative selection using MACS beads as described (see Kim, 2001, JCI 108:1331). Cells are then cultured for 4-6 days at 0.5×10⁶ cells/ml in complete RPMI (RPMI-1640+50 microg/50 U penicillin/streptomycin+10% FCS+50 microM beta-mercaptoethanol +1 mM sodium pyruvate +2 mM L-glutamine) in plates pre-coated 12 hr with 1-5 microg/ml anti-CD3 (Pharmingen). To these cultured T cells is added 1 microg/ml anti-CD28 antibody (Pharmingen) for co-stimulation and 5 ng/ml IL-2 for growth. Other reagents that can be substituted for co-stimulation include, but are not limited to, anti-CD49d, anti-CD2, or CD40-Ig at effective concentrations. In addition, cytokines important for the differentiation of T cells are added in particular combinations along with antibodies against other cytokines to induce differentiation and polarization. Useful combinations include 4 ng/ml IL-12, 10 ng/ml IFN-gamma, and 3 microg/ml anti-IL-4 to mimic TH1 differentiation; and 10 ng/ml IL-4, 3 microg/ml anti-IL-12, 3 microg/mI anti-IFN-gamma to mimic TH2 polarization conditions. In other embodiments, 10 ng/ml of IL-13, IL-6, or IL-9 may be added to the TH2 conditions, and 10 ng/ml IL-23 or IL-27 may be added to the TH1 conditions. Other polarization conditions include Tr1 polarization (10 ng/ml IL-10 and 4 ng/ml IFN-alpha_{2b}) or the neutral polarization (5 ng/ml IL-2 only). After 6 days, the same population of T cells may be re-stimulated in the same manner for another 6 days for further polarization.

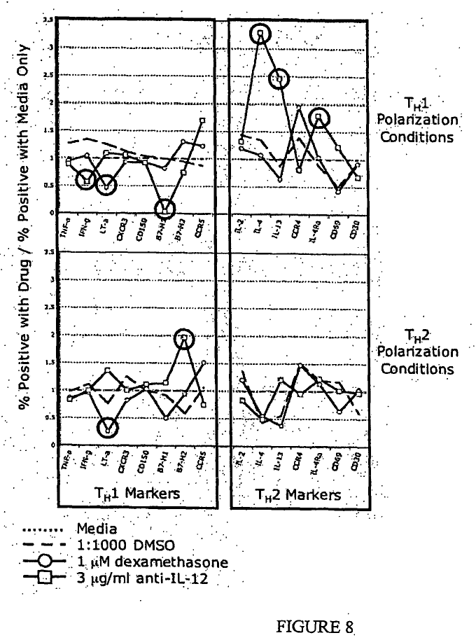

After the required time, T cells in the cultures are analyzed by flow cytometry for surface markers and intracellular cytokines. Anti-CD3 and anti-CD4 antibodies are used to identify the CD4+ T cells. Based on the parameters altered by the indicated differentiation conditions, BioMaps are generated for the parameters IFN-gamma, TNF-alpha, IL-2, IL-4, IL-13, LT-alpha, CCR4, CCR5, CXCR3, IL-4Ralpha, CD11c, CD134, CD150, CD137, CD69, B7-H1, B7-H2, and CD200. Other parameters of interest include alpha4beta7 integrin, L-selectin (CD62L), CCR7, CXCR5, CCR9, CCR2, cutaneous lymphocyte antigen (CLA), CTLA-4 (CD152) and CD154. Differences between TH1 and TH2 lymphocytes can be distinguished after both 6 and 12 days. See, for example, FIG. 8.

A database of BioMaps is generated from a panel of assay combinations that include the two polarization conditions (e.g. TH1 and TH2) and anti-inflammatory drug compounds. These compounds can include inhibitors of T cell activation and/or T cell proliferation such as the calcineurin inhibitors, FK-506 and cyclosporin A, and the proliferation inhibitors rapamycin, mycophenolic acid, and methotrexate. Other immuno-modulatory drugs (e.g. dexamethasone), or antibodies (e.g. anti-IL-12) can be screened and BioMaps generated that show the changes in the markers with the different agents. Such compounds include those described in The Pharmacologic Basis of Therapeutics.

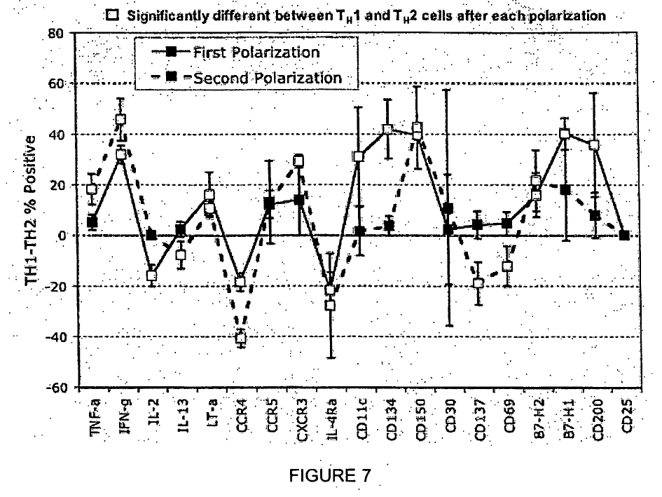

As shown in FIG. 7, BioMAP parameters are useful in characterizing TH1 or TH2 cells after one or two polarizations. A panel of markers (abscissa) of TH1 or TH2 differentiation were used to characterize the resulting T cell populations and determine the best parameters for identifying differences between TH1 and TH2 cells after each polarization.

FIG. 8 demonstrates the effects of drugs on TH1 and TH2 polarization. CD4+ peripheral blood T cells isolated by negative selection were polarized for two rounds under TH1 or TH2 conditions. During the second round of polarization, drugs or solvent controls were added to the culture every other day (days 1, 3, and 5). A panel of markers (abscissa of each plot) was used to characterize the TH1 or TH2 state of the CD4+ population after the second polarization. It may be noted that anti-IL-12 greatly impairs the ability of cells to polarize toward the TH1 state, but has little effect on the polarization toward the TH2 state since it is already present in the culture for TH2 polarization.

The BioMAPS with the known agents are compared with those for candidate test agents. This allows the recognition of the pathway(s) the candidate agent acts on, by comparing the changes in the level of the specific markers for known drugs affecting known pathways and the changes observed with the candidate agent. The database can also include reference BioMaps generated from assay panels containing cells with added genetic over-expression or knockdown constructs (e.g. constitutively active STAT5a*; FIG. 8) that selectively target or modulate specific cellular pathways (e.g. JAK/STAT, NF-AT, calcineurin, NF-kappaB, MAP kinase, and the like).

### EXAMPLE 7

### Reaulators of Monocyte Function

This Example illustrates how the methods of the present invention can be applied for the screening of compounds for modulating monocyte/macrophage function.

Human peripheral blood monocytes are used. Other cells that can be used in place human peripheral blood monocytes include bone-marrow derived monocytes, monocytes isolated by elutriation or negative magnetic bead isolation, and the monocyte cell lines THP-1 or U937. About 10×10⁶ peripheral blood mononuclear cells/ml are cultured in RPMI containing 10% fetal bovine serum for 1 hour. Non-adherent lymphocytes are removed by gentle washing.

The following are then applied for 5 or 24 hours: IL-1 (1 ng/ml), TNF-alpha (100 ng/ml), or LPS (200 ng/ml) (see Dietz, 1998, Basic Res. Cardiology 93 Suppl2:101; Lommi, 1997, Eur. Heart. J. 18:1620; and Jafri, 1997, Semin. Thromb. Hemost. 23:543). In subsequent panels, one or more of IFN-gamma (10 ng/ml), GM-CSF (10 ng/ml), IL-4 (20 ng/ml), IL-13 (30 ng/ml), IL-10 (10 ng/ml), osteopontin (10 ng/ml), thrombin (10 U/ml), CD40L, oxidized LDL (100 ug/ml), or minimally modified LDL are added to the initial three factors or may replace one of the three factors (see Brown, 2000, J. Clin. Endocrinol. Metab. 85:336; Ashkar, 2000, Science 287:860; de Boer, 1999, J. Pathol. 188:174; and Berliner, 1990, J. Clin. Invest 85:1260). Standard concentrations of agents are employed as described in the literature (Kaplanski, 1998, J. Immunol 158:5435, 1997; Hofman, Blood 92:3064; Li, 2000, Circulation 102:1970; Essler, 1999, JBC 274:30361; and Brown, 2000, J. Clin. Endocrinol. Metab. 85:336).

Based on the parameters altered by the indicated factors, BioMaps are generated. Illustrative parameters include Annexin V, TNF-alpha, IL-1-beta, IL-6, IL-8, MIP-1-alpha, Mac-1 (CD11b/CD18), IL-12, and MCP-1 (see Devaux, 1997, Eur. Heart J. 18:470; Kessler, 1998, Diabetes Metab. 24:327; Becker, 2000, Z. Kardiol. 89:160; Kaplanski, 1997, J. Immunol. 158:5435; and Li, 2000, supra). Other markers of interest that can be included in the BioMAP are CD14, PAI-1, urokinase-type plasminogen activator receptor (uPAR, CD87), IL-10, IL-18, tissue factor, fibrinogen-binding activity, MIG, TARC, MDC, RANTES, CD80, CD86, CD40 and CD36 (see Paramo, 1985, Br. Med. J. 291:573; Fukuhara, 2000, Hypertension 35:353; Noda-Heiny, 1995, Arterioscler. Thromb. Vasc. Biol. 15:37; de Prost, 1995, J. Cardiovasc. Pharmacol., 25 Suppl2:S114; van de Stolpe, 1996, Thromb. Haemost. 75:182; Mach, 1999, J. Clin. Invest. 104:1041; and Nicholson, 2000, Ann. N.Y. Acad. Sci. 902:128).

A database of BioMaps is generated from a panel of assay combinations that include known anti-atherogenic agents, including but not limited to statins, test compounds are screened, and a BioMap generated that shows the changes in the markers with the different test compounds. The BioMaps of the known agents are used to compare to candidate test agents. This allows the recognition of the pathway(s) on which the candidate drug act, as determined by comparing the changes in the level of the specific markers for known drugs affecting known pathways and the changes observed with the candidate test compound. The database reference BioMaps can include those generated from assay panels containing cells with genetic constructs that selectively target or modulate specific cellular pathways (e.g. NFKB, MAP kinase, and the like), or cells that contain known genetic mutations (e.g. CD36-deficiency, see Yanai, 2000, Am. J. Med. Genet. 93:299, and the like).

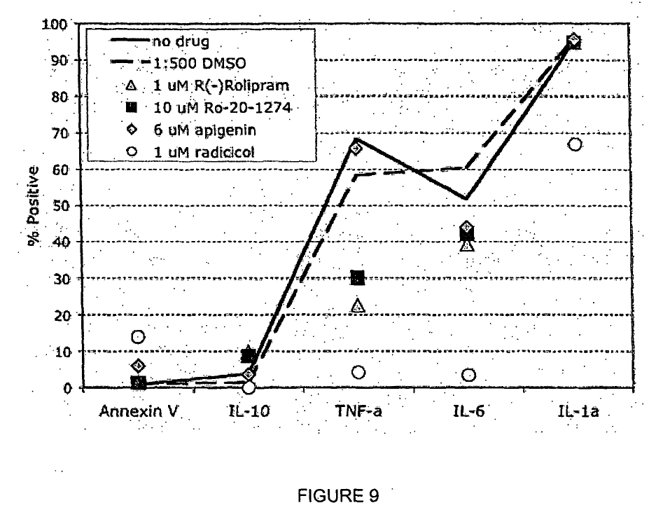

As shown in FIG. 9, BioMap analysis can be used to characterize the effects of drugs on LPS-stimulated monocyte cytokine secretion. CD14+ monocytes were enriched by adherence to 24-well plastic tissue culture dishes for 1 hr and unbound cells were removed. Monocytes were stimulated with 1 μg/ml LPS for 5 hr in the presence of 2 μM monensin (a secretion inhibitor). Drugs or solvent control (DMSO) at the indicated concentrations were added 15 minutes before LPS stimulation and were present for the entire 5 hr. Annexin V staining indicates apoptotic cells. Intracellular cytokines were detected by fixing and permeabilizing the cells after scraping them off of the culture dish.

What is claimed is:

1. A method for determining the activity of a biologically active agent according to its effect on cellular signaling pathways, the method comprising:
contacting a test biologically active agent with a cell culture system comprising primary human cells simultaneously activated in multiple signaling pathways;
recording changes in at least two different cellular parameter readouts after introduction of said agent;
deriving a first biomap dataset from said changes in parameter readouts wherein said biomap comprises data normalized to control data on the same said primary human cells simultaneously activated in multiple signaling pathways under control conditions lacking said biologically active agent, and wherein output parameters are optimized so that the biomap dataset is sufficiently informative that it can discriminate the mode of action or functional effect of an agent;
comparing said first biomap dataset to a reference biomap dataset to determine the presence of variation, wherein the presence of variation indicates a difference in the effect of the test biologically active agent on a cellular signaling pathway.

2. The method according to claim 1, wherein said cell culture system comprises at least two different factors.

3. The method according to claim 1, wherein said cell culture system comprises at least three different factors.

4. The method according to claim 1, wherein said cell culture system comprises multiple human cell types.

5. The method according to claim 1, wherein said primary human cells are endothelial cells.

6. The method according to claim 5, wherein said cell culture system comprises multiple human cell types.

7. The method according to claim 6, wherein said cell culture system comprises endothelial cells in combination with peripheral blood mononuclear cells, or a subset thereof.

8. The method according to claim 6, wherein said cell culture system comprises endothelial cells in combination with blood polymorphonuclear cells or a subset thereof.

9. The method according to claim 1, wherein at least four parameters and not more than ten parameters are measured.

10. The method according to claim 1, further comprising the steps of:
contacting said test biologically active agent with a second cell culture system comprising primary human cells simultaneously activated in multiple signaling pathways;
recording changes in at least two different cellular parameter readouts after introduction of said agent;
deriving a second biomap dataset from said changes in parameter readouts wherein said biomap comprises data normalized to control data on the same said primary human cells simultaneously activated in multiple signaling pathways under control conditions lacking said biologically active agent, and wherein output parameters are optimized so that the biomap dataset is sufficiently informative that it can discriminate the mode of action or functional effect of an agent;
comparing said first and said second biomap dataset to a reference biomap dataset to determine the presence of variation, wherein the presence of variation indicates a difference in the effect of the test biologically active agent on a cellular signaling pathway.

11. The method according to claim 10, wherein biomap dataset is ordered in a correlation plot by multidimensional scaling.

12. The method according to claim 10, wherein said comparing step comprises objective evaluation of the significance of all pairwise correlations between agent activities.
